(19) United States
(12) Patent Application Publication
(10) Pub. No.: US 2026/0053437 A1
(43) Pub. Date: Feb. 26, 2026

(54) MEDICATION DOSAGE RECOMMENDATIONS USING CHEMICAL SENSORS

(71) Applicant: Cardiac Pacemakers, Inc., St Paul, MN (US)

(72) Inventors: Pramodsingh Hirasingh Thakur, Woodbury, MN (US); Michael J. Kane, St Paul, MN (US); Ramesh Wariar, Blaine, MN (US); Yingbo Li, Woodbury, MN (US)

(21) Appl. No.: 19/305,037

(22) Filed: Aug. 20, 2025

Related U.S. Application Data
(60) Provisional application No. 63/685,257, filed on Aug. 20, 2024.

(51) Int. Cl. A61B 5/00; A61B 5/145; A61B 5/1473

(52) U.S. Cl. CPC A61B 5/4848; A61B 5/1451; A61B 5/14546; A61B 5/1473; A61B 5/6848; A61B 5/7275; A61B 5/742

(57) ABSTRACT

Systems, devices, and methods include approaches involving monitoring one or more medication dosages; estimating an analyte level using a chemical sensor that is part of an implantable device or a wearable device; determining an analyte response to the one or more medication dosages; and, after the determining, adjusting a medication dose regimen.

### CROSS REFERENCE TO RELATED APPLICATION

This application claims priority to Provisional Application No. 63/685,257, filed Aug. 20, 2024, which is herein incorporated by reference in its entirety.

### TECHNICAL FIELD

Instances of the present disclosure relate to using analyte sensing technology for evaluating and adjusting medication dose regimens.

### BACKGROUND

Medications can affect a person's analyte levels. Some medications can positively impact levels one type of analyte but negatively impact levels of another type of analyte.

### SUMMARY

In Example 1, a method includes monitoring one or more medication dosages, estimating an analyte level using a chemical sensor (e.g., based on an optical property of a chemical sensor) that is part of an implantable device or a wearable device, determining an analyte response to the one or more medication dosages, and after the determining, adjusting a medication dose regimen.

In Example 2, the method of Example 1, wherein the chemical sensor is part of the wearable device, which includes needles sized for access to interstitial fluid and a chemical indicator positioned within the needles.

In Example 3, the method of Example 2, wherein the optical property of the chemical indicator changes in response to different analyte levels.

In Example 4, the method of any of Examples 1-3, further including determining the analyte level based, at least in part, on the optical property contained in a digital image.

In Example 5, the method of Example 1, wherein the chemical sensor is part of the implantable device.

In Example 6, the method of any of Examples 1-5, wherein the one or more medication dosages includes a first set of dosages from a first regimen and a second set of dosages from a second regimen, wherein the determining the analyte response comprises determining a difference between the analyte response to the first regimen and the second regimen.

In Example 7, the method of any of Examples 1-6, wherein the one or more medication dosages involve a first type of medication, wherein the medication dose regimen comprises adding a second type of medication.

In Example 8, the method of Example 7, wherein the second type of medication is a potassium binder medication or a potassium supplement medication.

In Example 9, the method of any of Examples 1-8, wherein the adjusting a medication dose regimen comprises adjusting a timing between medication doses.

In Example 10, the method of any of Examples 1-8, wherein the adjusting a medication dose regimen comprises adjusting a dosage amount of each dose.

In Example 11, the method of any of Examples 1-10, wherein the adjusting the medication dose comprises recommending a different time of day to take the medication.

In Example 12, the method of any of Examples 1-11, wherein the analyte level is a potassium level.

In Example 13, a computer program product comprising instructions to cause one or more processors to carry out the steps of the method of Examples 1-12.

In Example 14, a computer-readable medium having stored thereon the computer program product of Example 13.

In Example 15, a mobile device comprising the computer-readable medium of Example 14.

In Example 16, a system includes a mobile computing device with a processor, memory, and a user interface. The mobile computing device is programmed to: estimate an analyte level using a chemical sensor (e.g., based on an optical property of a chemical sensor) that is part of an implantable device or a wearable device, determine an analyte response to the one or more medication dosages, and adjust a medication dose regimen based, at least in part, on the analyte response.

In Example 17, the system of Example 16, further comprising: the wearable device with a set of needles sized for access to interstitial fluid and a chemical indicator positioned within the set of needles.

In Example 18, the system of Example 17, wherein the mobile computing device includes an image sensor.

In Example 19, the system of Example 18, wherein the mobile computing device is programmed to: determine the analyte level based, at least in part, on the optical property of the chemical indicator in a digital image.

In Example 20, the system of Example 17, wherein the chemical indicator changes optical properties in response to different potassium levels.

In Example 21, the system of Example 16, further comprising: the implantable medical device comprising a chemical indicator that changes the optical property in response to different analyte levels.

In Example 22, the system of Example 16, wherein adjusting the medication dose regimen comprises displaying a new medication dose regimen on the user interface.

In Example 23, the system of Example 16, wherein adjusting the medication dose regimen comprises changing a dosage amount of each dose.

In Example 24, a system including a mobile computing device with a processor, memory, and a user interface. The mobile computing device is programmed to: monitor analyte levels using a chemical sensor (e.g., based on an optical property of a chemical sensor) that is part of an implantable device or a wearable device, determine an analyte time trend based, at least in part, on the analyte levels, adjust a time of day for taking a medication dose based, at least in part, on the analyte time trend, and display the time of day for taking the medication dose on the user interface.

In Example 25, a method includes monitoring one or more medication dosages, estimating an analyte level using a chemical sensor (e.g., based on an optical property of a chemical sensor) that is part of an implantable device or a wearable device, determining an analyte response to the one or more medication dosages, and adjusting a medication dose regimen based, at least in part, on the analyte response.

In Example 26, the method of Example 25, wherein the one or more medication dosages includes a first set of dosages from a first regimen and a second set of dosages from a second regimen, wherein the determining the analyte response comprises determining a difference between the analyte response to the first regimen and the second regimen.

In Example 27, the method of Example 25, wherein the one or more medication dosages involve a first type of medication, wherein the adjusting the medication dose regimen comprises adding a second type of medication.

In Example 28, the method of Example 27, wherein the second type of medication is a potassium binder medication or a potassium supplement medication.

In Example 29, the method of Example 28, wherein the analyte level is a potassium level.

In Example 30, the method of Example 25, wherein the adjusting a medication dose regimen comprises adjusting a timing between medication doses.

In Example 31, the method of Example 25, wherein the adjusting a medication dose regimen comprises adjusting a dosage amount of each dose.

In Example 32, the method of Example 25, wherein the adjusting the medication dose comprises recommending a different time of day to take the medication.

In Example 33, the method of Example 25, wherein the chemical sensor is part of the wearable device, which includes needles sized for access to interstitial fluid and a chemical indicator positioned within the needles.

In Example 34, the method of Example 33, wherein the optical property of the chemical indicator changes in response to different analyte levels.

In Example 35, the method of Example 25, wherein the chemical sensor is part of the implantable device.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows a block diagram of a method for use with one or more components of a chemical sensing system, in accordance with certain instances of the present disclosure.

FIG. 3 shows a schematic illustration of a computing device, in accordance with certain instances of the present disclosure.

FIG. 4 shows a graph of a person's analyte levels over time, in accordance with certain instances of the present disclosure.

FIG. 5 shows a block diagram of a method for use with one or more components of a chemical sensing system, in accordance with certain instances of the present disclosure.

FIG. 6-8 show different views of various portions of a wearable chemical sensing device, in accordance with certain instances of the present disclosure.

FIGS. 9 and 10 show different views of various portions of an implantable medical device, in accordance with certain instances of the present disclosure.

FIG. 11 shows a block diagram with additional details of the computing device of FIG. 3, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Certain analytes (e.g., potassium, sodium, creatinine) can be measured and monitored to evaluate kidney and/or cardiac conditions and performance.

A person's analyte levels (e.g., concentrations) will naturally modulate over a 24-hour-day period, and analyte levels can be affected by a person's diet, exercise, and medication.

Certain instances of the present disclosure are accordingly directed to approaches (e.g., systems, methods, devices) that use analyte sensing technology for evaluating analyte levels and making medication dose regimen adjustments—such as the timing, dosage amounts, and types of medication.

Particular instances of the present disclosure are focused on measuring, monitoring, and using potassium levels (e.g., potassium concentrations) to make medication dose regimen adjustments. Some medications that are prescribed for heart failure (e.g., ACE inhibitors, angiotensin receptor blockers, mineralocorticoid receptor antagonists, angiotensin receptor neprilysin inhibitors) impact how a person's renin-angiotensin-aldosterone system (RAAS) functions, including their kidneys'sodium-potassium exchanger (e.g., Na+ K+ pump). This can result in sodium levels and/or glucose levels being reduced but potassium levels increasing.

Increased levels of potassium can negatively impact a person's cardiac rhythm (e.g., the heart's electrical activity such as its excitation cycle). The excitation cycle of cardiac cells is influenced by the cardiac cells'resting electrical potential and by the activity of ion channels (such as potassium, sodium, and calcium ion channels) in the cell membrane of the cardiac cells. When the concentration of potassium in plasma is within a normal range, the potassium ion channels can function effectively. However, when the potassium concentration in the plasma is elevated (referred to as “hyperkalemia”), the concentration gradient of potassium across the cardiac cell membrane is reduced and the cardiac cell generally becomes depolarized and in-excitable. In contrast, when the potassium concentration is low (referred to as “hypokalemia”), the concentration gradient of potassium across the cardiac cell membrane is increased resulting in hyperpolarization of the resting electrical potential. Hypokalemia can lead to arrhythmias, such as atrial fibrillation.

Certain oncology medications (e.g., methotrexate, ifosfamide, cisplatin) have renal side effects including disrupting how a person's kidneys process pH, glucose, or potassium.

### Analyte Sensing System

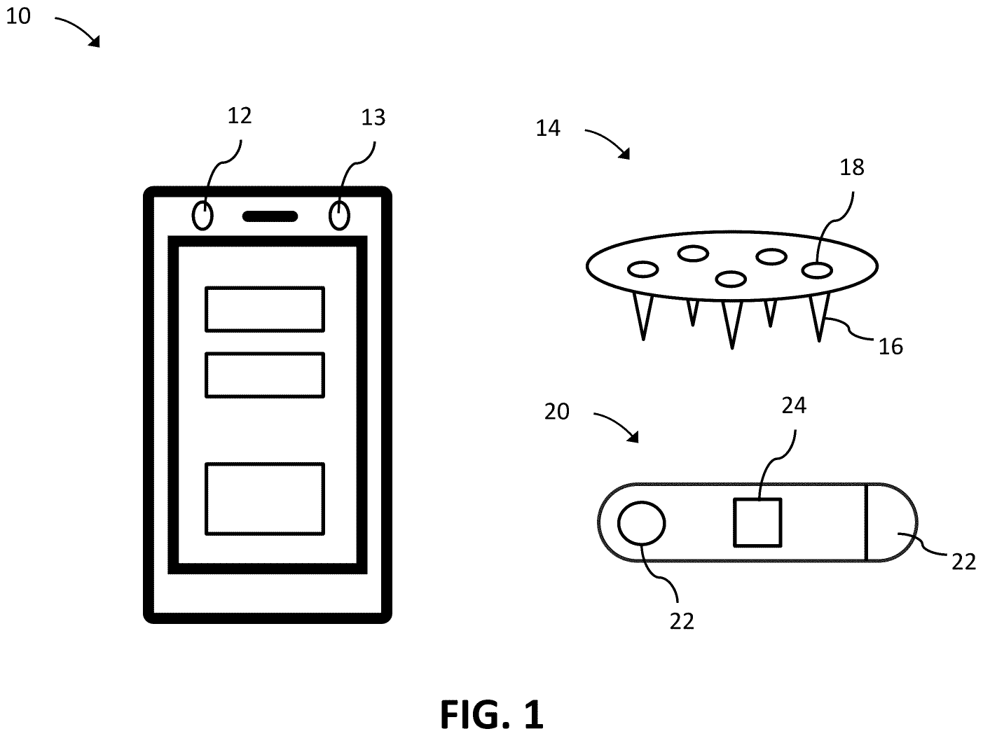

FIG. 1 shows a chemical sensing system 10 (hereinafter “the system 10” for brevity) with schematic representations of two sets of components that can be used to measure analyte concentrations. Typically, measuring a patient's analyte concentrations requires drawing multiple blood samples from a patient at a clinic and processing the blood samples at a laboratory. Components of the chemical sensing system 10 can be used to measure analyte concentrations using a wearable device or an implantable medical device. Although the chemical sensors shown in FIG. 1 and described below are optical-based chemical sensors, other types of chemical sensor approaches can be used (e.g., electrical-based chemical sensors such as those that use electrodes and measure potential differences, and the like).

For the wearable approach, the system 10 can include a device (e.g., a mobile computing device described further herein) with an image sensor 12 and a chemical sensing device 14. The image sensor 12 (e.g., a charge coupled device, a complementary metal oxide semiconductor, or other devices that can capture an image) can be part of a camera, smart phone, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a mobile computing device such as a smart phone, the smart phone can store, operate, or otherwise access a program (e.g., a phone application) that processes an image (of the chemical sensing device 14) taken by the image sensor 12 and determines estimates of one or more analyte concentrations of the patient. In other instances, the image sensor 12 is part of a dedicated readout device or part of a camera. The system 10 can include one or more light sources 13, which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes a mechanism such as multiple needles 16 (e.g., microneedles) for access to a patient's interstitial fluid. For example, the needles 16 can be sized to access a patient's interstitial fluid. The patch 14 can also include multiple chemical indicators 18, each of which changes optical properties (e.g., fluorimetric properties, colorimetric properties) with changes in concentration of a certain analyte in the interstitial fluid. As described in more detail herein, the image sensor 12 can be used to capture an image (e.g., a digital image) of the chemical indicators 18, and the image can be processed and analyzed to determine respective concentrations of targeted analytes. In certain instances, the patch 14 includes one type of chemical indicator 18 (e.g., to help determine concentration of one type of analyte), but in other instances the patch 14 includes multiple types of chemical indicators.

For the implantable approach, the system 10 can include an implantable medical device 20, which includes one or more electrodes 22 and a chemical sensor assembly 24. The electrodes 22 can comprise a conductive material and be configured to sense cardiac activation signals. The chemical sensor assembly 24 can include a sensing element with a polymeric matrix permeable to analytes such as creatinine and/or potassium. The sensing element can include an interior volume with various chemical indicators (e.g., beads for detecting an ion concentration of a bodily fluid when implanted in the body disposed within an interior volume). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels. The estimated analyte levels can be used by a computing device to monitor and evaluate a person's kidney and/or cardiac performance.

### Methods

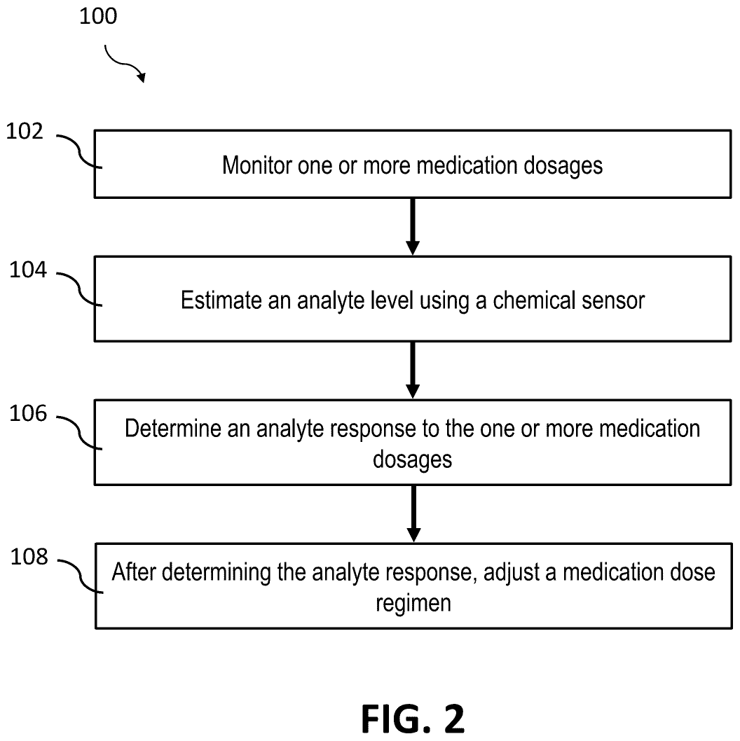

FIG. 2 outlines a method that can be used in connection with one or more components of the system 10 of FIG. 1 or other components described herein.

FIG. 2 shows a block diagram of a method 100 for making medication dose regimen adjustments. In certain instances, the method 100 is carried out by one or more computing devices such as a mobile phone, a tablet, a laptop computer, a desktop computer, a server. For example, the method 100 could carried out by an application (e.g., a phone app) that is downloadable to the device, operated on the device, and accessible by a patient, their physician, their clinic, and the like.

The method 100 includes monitoring one or more medication dosages (block 102 in FIG. 2). When a person is prescribed and begins to take a medication, the medication doses can be monitored. Monitoring the medication doses can include monitoring the amount of medication taken (e.g., individual does amounts, cumulative dosages over time), the time of day each dose was taken, and the like. In some instances, information about the medication doses is manually inputted by the person taking the medication, and in other instances, information about the medication doses is automatically generated.

As one example, a person taking the medication can input the dosage amount and the time taken for each medication dose. In some instances, the person inputs the dosage information into a user interface of a computing device (e.g., via an application on their mobile phone, tablet). In other instances, the person inputs the dosage amount via a voice command to a computing device such as their mobile phone or smart speaker.

As another example, if the medication is dispensed automatically, the device dispensing the medication can track the dosage amount and the time taken for each medication dose. In some instances, the medication dose is dispensed by a computing-device-controlled pill dispenser, and therefore the pill dispenser can track the medication doses. In some instances, the medication dose is dispensed by a computing-device-controlled injector (e.g., a subcutaneous injector using a pump or other mechanism), and therefore the injector can track the medication doses.

The method 100 further includes estimating an analyte level using a chemical sensor (e.g., an analyte level based on an optical property) that is part of an implantable device or a wearable device (block 104 in FIG. 2). In certain instances, the analyte level being measured is a potassium level. The chemical sensor can be the types of chemical sensors described in connection with FIG. 1 and herein. A chemical indicator that is part of the chemical sensor can be in communication with a person's blood such that one or more optical properties of the chemical indicator can be monitored to determine a level or concentration of the analyte.

In certain instances, estimating an analyte level occurs periodically (e.g., every 30 minutes, once an hour) or on demand (e.g., when a patient or physician initiates the comparison). Although the chemical sensors may react in real-time (e.g., the chemical indicators change optical properties in real-time as analyte levels change in real-time), transmission of or calculating an analyte level and comparing the analyte level to the threshold less often can save computing and battery resources and may be preferable because analyte levels may not change drastically minute-by-minute.

The method 100 further includes determining an analyte response to the one or more medication dosages (block 106 in FIG. 2). In some instances, determining the analyte response involves comparing how different medication regimens affect analyte levels. For example, analyte levels can be monitored while a person follows a first medication regimen (e.g., a regiment that may include a specific number, amount, and timing of medication doses over a period of time). Then, analyte levels can be monitored while a person follows a second medication regimen (and third medication regimen, fourth medication regiment, etc.). The difference between the analyte response to the different medication regimens can then be determined. For example, the analyte response can be a relationship that specifies how a given change (e.g., a difference) in a medication dose (e.g., amount in milligrams) results in a given change in levels of the analyte (e.g., in terms of mEq/L). As a more specific example, when monitoring potassium levels, the analyte response can be a Δ dose to Δ potassium relationship that can be used to determine how a change in a medication dose regimen will cause a change in potassium levels. Similar dose-to-analyte relationships can be determined when monitoring other analytes.

The method 100 further includes—after determining the analyte response—adjusting a medication dose regimen (block 108 in FIG. 2). There are several different approaches for adjusting the medication dose regimen. These different approaches are not mutually exclusive. As such, more than one approach can be used at a time to adjust the medication dose regimen. Further, in certain instances, adjusting the medication dose regimen involves adjusting what medication dose regimen is recommended to a person. For example, if a user interface is used to display information about medication dose regimens such as recommended timing, dose amount, etc., adjusting the medication dose regimen can include adjusting the recommendations displayed on the user interface. In certain instances, adjusting the medication dose regimen is based, at least in part, on predictions of future analyte levels and avoiding future analyte levels crossing one or more thresholds.

In one example, adjusting the medication dose regimen involves adjusting a timing between medication doses. If the prior medication dose regimen causes an increase in analyte levels, the timing between doses can be increased and vice versa. For potassium, serum potassium levels indicate the amount of potassium in the blood, and normal serum potassium levels for adults range from 3.5-5.0 mEq/L. As such, if a prior medication dose regimen causes potassium levels to reach above 5.0 mEq/L or below 3.5 mEq/L (or some other threshold), the timing between medication doses can be adjusted. As noted above, adjusting can involve adjusting the recommended timing displayed on a user interface.

In another example, adjusting the medication dose regimen involves adjusting a dosage amount of one or more doses in the medication dose regimen. If the prior medication dose regimen causes an increase in analyte levels above a threshold, the dosage amount of one or more doses can be decreased. As noted above, adjusting can involve adjusting the recommended dosage amount displayed on a user interface.

In another example, adjusting the medication dose regimen involves adjusting the time for taking one or more doses of the medication dose regimen. This approach may involve determining or predicting what time a person's analyte level is (or is likely to) be at or near its lowest level. The medication dose regimen can be adjusted such that one or more doses are taken during periods with relatively lower analyte levels. As noted above, adjusting can involve adjusting the recommended time to take one or more medication doses as displayed on a user interface.

In another example, adjusting the medication dose regimen involves adjusting the type of medication being taken. Using potassium as an example, if a person's potassium levels trend higher with a first type of medication, a potassium-binder medication can be used in the medication dose regimen. If a person's potassium levels trend lower with a first type of medication, a potassium-supplement medication can be used in the medication dose regimen. Similar approaches can be used when monitoring other types of analytes. The second type of medication can be used in place of or in addition to the first type of medication. As noted above, adjusting can involve adjusting the recommended medication type as displayed on a user interface. Additionally or alternatively, if analyte levels trend higher or lower, a different diet can be recommended to the person.

In another example, adjusting the medication dose regimen involves delaying or suspending the next dose or set of doses. For example, if the person's analyte levels are above a threshold (or predicted to be above a threshold), subsequent medication doses can be delayed or suspended to avoid reaching or maintaining unhealthy analyte levels.

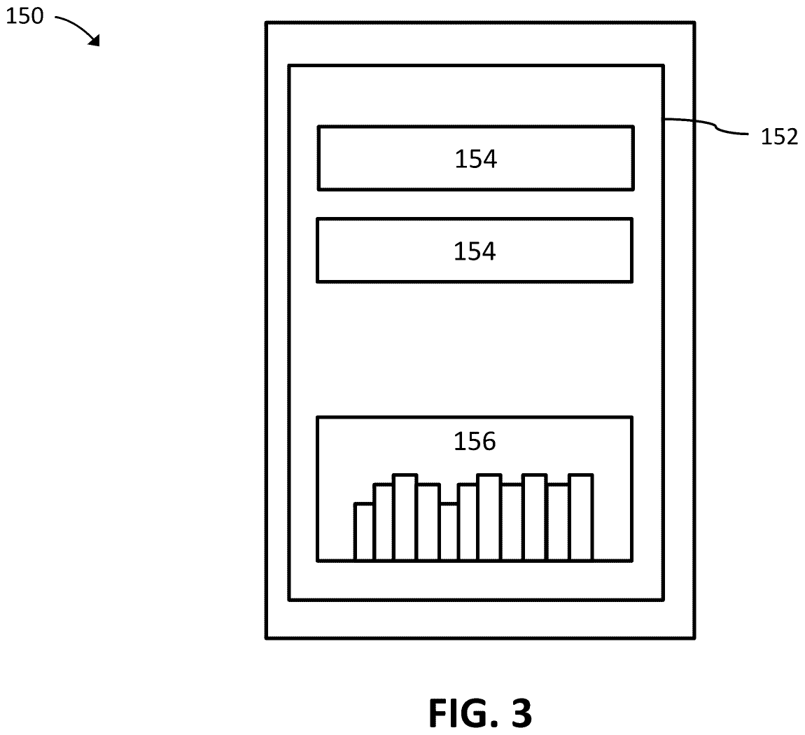

In certain embodiments, the medication dose regiment can be displayed visually on a user interface of a computing device (e.g., a mobile computing device). FIG. 3 shows an example computing device 150 such as a mobile phone or tablet that includes a user interface 152. The device 150 can operate an application such that the user interface 152 displays various screens, icons, and/or buttons to help the user navigate and use the application. One or more graphics, pictures, videos, and/or text passages 154 can be used to display various aspects (e.g., recommendations) relating to the medication dose regimen. Any of the data described herein—whether historical or in real-time—can be displayed on the user interface 152 of the computing device 150. For example, the user interface 152 can be arranged to include a window 156 in which one or more sets of data can be displayed (e.g., analyte levels as a function of time, medication dosage amounts and timing).

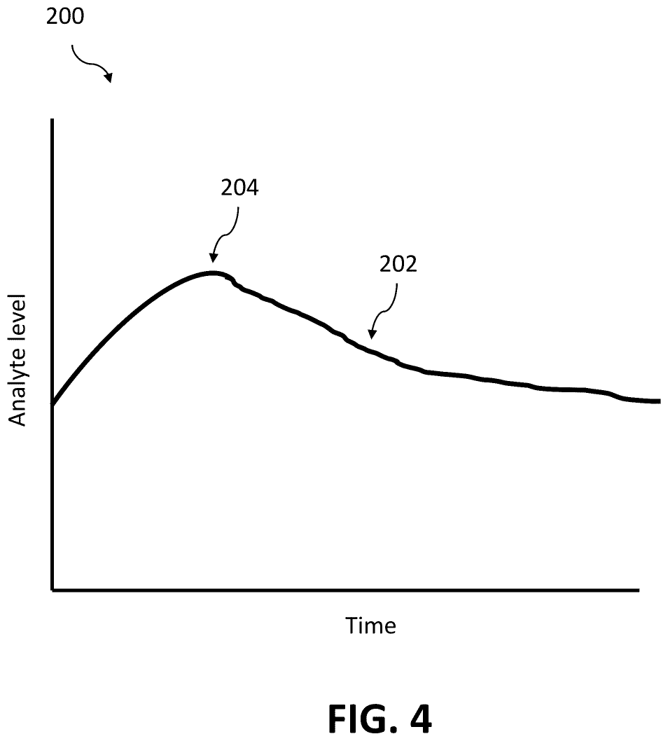

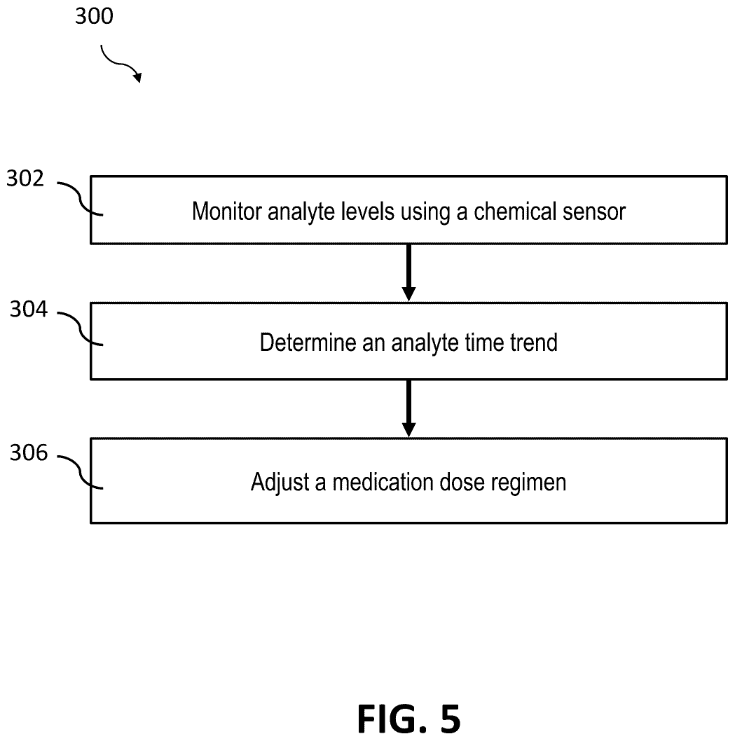

FIG. 4 shows a graph 200 of a person's analyte levels over time, and FIG. 5 outlines a method that can be used in connection with analyte level trends over time. The graph 200 includes a plot 202 of a person's analyte level such as potassium as a function of time.

In some instances, the plot 202 represents a time period after the person has taken a medication dose. As noted above, medication can increase analyte levels such as potassium levels. A person's analyte response can be determined such that the response can be used to estimate when a person's analyte level will peak 204 after having taken a medication dose. The analyte response can then be used to adjust the medication dose regimen to help prevent analyte levels crossing a maximum threshold, for example.

In other instances, the plot 202 represents a person's natural variation of analyte levels throughout the day, where analyte levels modulate between peaks and lulls. Typically, analyte levels peak near the middle of the day and hit a low point at night. A person's analyte levels can be monitored such that time-based trends can be determined and used to adjust medication dose regimens.

FIG. 5 outlines a method that can be used in connection with one or more components of the system 10 of FIG. 1 or other components described herein.

FIG. 5 shows a block diagram of a method 300 for making medication-related recommendations based on a historical analyte levels and/or response to one or more medication doses. In certain instances, the method 300 is carried out by one or more computing devices such as a mobile phone, a tablet, a laptop computer, a desktop computer, a server. For example, the method 300 could carried out by an application (e.g., a phone app) that is downloadable to the device, operated on the device, and accessible by a patient, their physician, their clinic, and the like.

The method 300 includes monitoring analyte levels (e.g., analyte levels based on an optical property) using a chemical sensor that is part of an implantable device or a wearable device (block 302 in FIG. 5). In certain instances, the analyte level being measured is a potassium level. The chemical sensor can be the types of chemical sensors described in connection with FIG. 1 and herein. A chemical indicator that is part of the chemical sensor can be in communication with a person's blood such that one or more optical properties of the chemical indicator can be monitored to determine a level or concentration of the analyte.

The method 300 further includes determining an analyte time trend (block 304 in FIG. 5). In some instances, the analyte time trend includes a person's analyte response to taking one or more medication dosages. Additionally or alternatively, the analyte time trend includes a person's typical analyte level at various times throughout a 24-hour period.

The method 300 further includes adjusting a medication dose regimen (block 306 in FIG. 5). In particular, adjusting the medication dose regimen can include adjusting a time of day when a medication dose is taken or recommended to be taken.

The method 300 can further include—after calculating the recommended adjustment—generating a visual recommendation on a user interface that displays the recommended adjustment to the medication dose regimen.

### Wearable Chemical Sensor

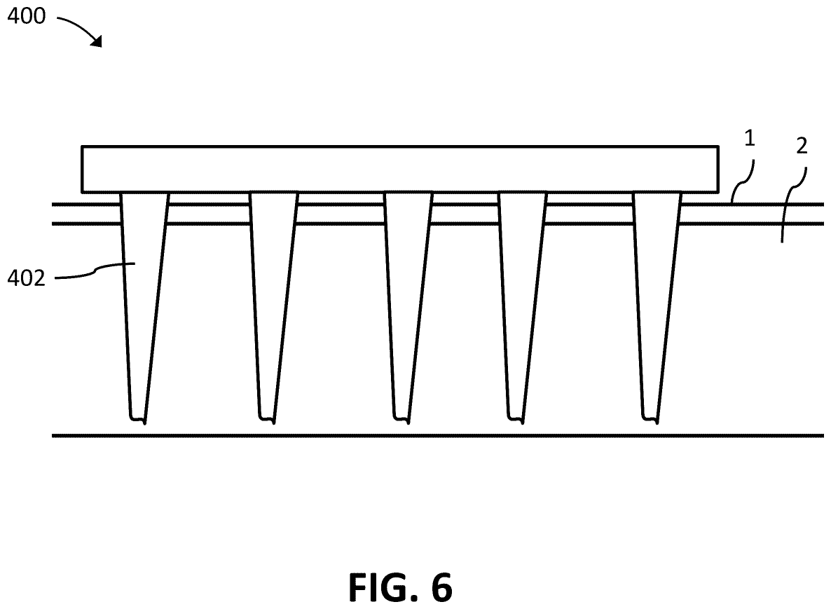

FIG. 6 shows a schematic side view of a wearable chemical sensing device 400. For simplicity, the device 400 is hereinafter referred to as the “patch 400” although other types of wearable devices can use the chemical sensing technology described herein. The patch 400 can be coupled to the patient's skin 1 such that needles 402 pierce through the outer layer of skin 1 and extend into the patient's interstitial fluid space 2. The needles 402 can have openings that are exposed to the patient's interstitial fluid (and therefore analytes within the patient's interstitial fluid).

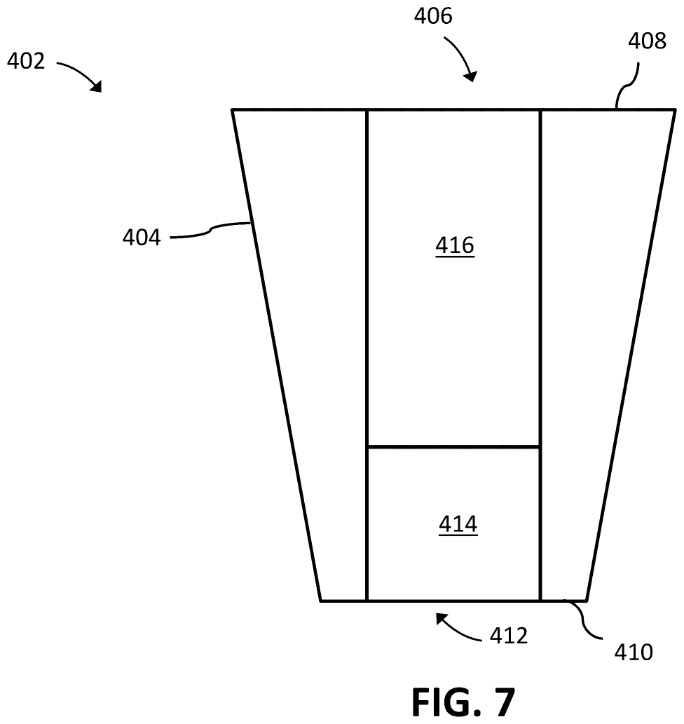

FIG. 7 shows a schematic side view of one of the needles 402 of the patch 400. In certain instances, the needles 402 are hollow needles such that each needle 402 includes an outer needle structure 404 that surrounds an opening 406 (e.g., a central thru-hole within the needle 402). The opening 406 can extend from a proximal end 408 of the needle 402 to a distal end 410 of the needle 402. An aperture 412 is located at or near the distal end 410 of the needle 402 such that the opening 406 is exposed to interstitial fluid.

Also at or near the distal end 410 of the needle 402 is a membrane 414 (e.g., a diffusion membrane) that is positioned within the needle 402. The membrane 414 protects tissue from direct interaction or exposure to a chemical indicator 416 that is also positioned within the needle 402. The membrane 414 can be formed from a permeable material, such as an ion permeable polymeric matrix material. In some instances, the membrane 414 can be permeable to sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 414 includes an active agent disposed therein including, but not limited to anti-inflammatory agents, angiogenic agents, and the like.

The particular type (e.g., type of ion selectivity) and length of membrane can vary by needle 402. For example, one set of needles 402 can include a membrane 414 that is permeable to creatinine ions, while another set of needles 402 includes a membrane 414 that is permeable to potassium or sodium ions, and so on. In other examples, the membrane 414 is agnostic to a particular type of ion. The membrane 414 is positioned such that analytes must pass through the membrane 414 before reaching the chemical indicator 416. The membrane 414 material used will affect how fast an analyte travels between interstitial fluid and the chemical indicator 416.

The chemical indicator 416 comprises a material that changes properties (e.g., optical properties such as absorption, transmission, scattering, fluorescence) with changes in concentration of a given analyte. As one example, the chemical indicator 416 can comprise a creatinine select compound that changes optical properties in response to the creatinine select compound binding to creatine. As another example, the chemical indicator 416 can comprise a creatinine deiminase enzyme covalently bound to a substrate and a pH-indicating compound in ionic communication with the creatinine deiminase enzyme. In this example, the chemical indicator 416 can change optical properties in response to changes in creatinine concentrations in vivo. As another example, the chemical indicator 416 can comprise a creatinine select compound, and a pH-indicating compound in ionic communication with bodily fluid. In this example, the chemical indicator 416 can change optical properties in response to changes in creatinine concentrations in vivo. And in this example, the chemical indicator 416 may also comprise a mechanism to change local pH within the chemical indicator 416.

In certain instances, color of the chemical indicator 416 comprises the sum of the absorption, transmission, reflectance, and fluorescence properties of the chemical indicator material. Put another way, the chemical indicator 416 can comprise a material that changes optical properties with changes in concentration of a given analyte—and such optical properties can be measured by analyzing an image of the chemical indicator 416. In certain instances, the chemical indicator 416 has a minimum thickness or height along a longitudinal axis of a needle of 0.15-0.60 mm (e.g., 0.50-0.60 mm). In certain instances, the chemical indicator 416 comprises a slurry or a film.

In certain instances, the chemical indicator 416 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 416 can include components (or response elements) that are configured for a colorimetric response, a photoluminescent response, or another optical sensing modality. For example, the chemical indicator 416 can include an element that changes color based on binding with or otherwise complexing with a specific chemical analyte. As one specific example, creatinine reacts with a molecule which changes pH and color on the indicator. In some instances, the chemical indicator 416 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 416 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy)furyl]coumarin. Sodium specific fluoroionophores can include (6,7-[2.2.1]-cryptando-3-[2″-(5″-carboethoxy)furyl]coumarin. Potassium specific fluoroionophores can include (6,7-[2.2.2]-cryptando-3-[2″-(5″-carboethoxy)furyl]coumarin and (6,7-[2.2.2]-cryptando-3-[2″-(5″-carboethoxy)thiophenyl]coumarin.

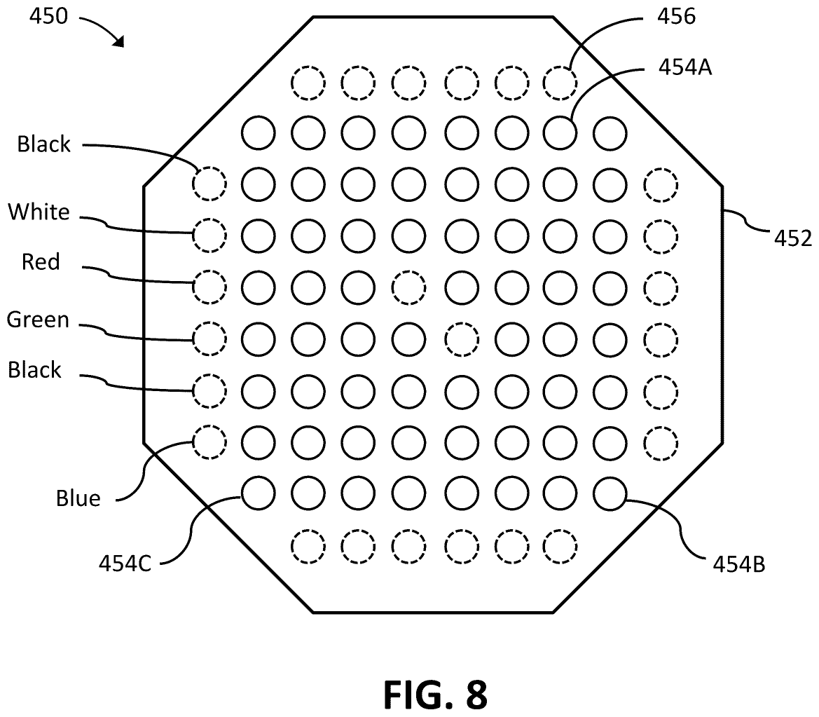

FIG. 8 shows a top view of a patch 450. The arrangement shown in FIG. 8 can be used in connections with the needles, membranes, chemical indicators, and layers, etc., described above with respect to the patch 400. The view shown in FIG. 8 is the type of view of a patch that an image sensor would capture in a digital image while the patch is coupled to the patient. The digital image can capture the colors of the chemical indicators (and color references) such that the colors can be analyzed to determine concentrations of one or more analytes of the patient.

FIG. 8 shows a patch 450, which includes a window 452 through which various components of the patch 100 can be viewed. In particular, the window 452 allows chemical indicators 454A-C of the patch 450 to be viewed. The chemical indicators 454A-C can be positioned in needles (e.g., hollow needles) such as the needles described above. As such, each chemical indicator 454A-C can be associated with its own needle.

A first set of needles can include a first type of chemical indicator 454A such as a chemical indicator that changes in color with changes in concentration of a first analyte (e.g., creatinine). A second set of needles can include a second type of chemical indicator sodium 454B such as a chemical indicator that changes in color with changes in concentration of a second analyte (e.g., potassium). A third set of needles can include a third type of chemical indicator 454C such as a chemical indicator that changes in color with changes in concentration of a third analyte (e.g., sodium). The respective colors of the chemical indicators can be used to estimate the respective concentrations of analytes in a patient's interstitial fluid.

In certain instances, each of the first type of chemical indicators 454A are positioned near or next to each other, each of the second type of chemical indicators 454B are positioned near or next to each other, and so on. The overall number of chemical indicators (and therefore the number of needles) and the number of different sets of types of chemical indicators on a given patch can be fewer or greater than that shown in FIG. 8. For example, the patch 450 could include a single type of chemical indicator selected for a single type of analyte. The relative positions of the chemical indicators 454A-C can vary from that shown in FIG. 8, and the specific shape of the chemical indicators 454A-C (as seen from a top view) can vary from the circular shapes shown in FIG. 8.

The patch 450 can also include color references 456. The color references 456 are shown in dotted lines in FIG. 8. The color references 456 can help with calibrating, correcting, and/or processing the digital image of the chemical indicators 454A-C such that an accurate estimate of the color of the chemical indicators 454A-C can be determined. For example, because the color of the color references 456 is known, the color of the chemical indicators 454A-C can be more accurately estimated as the patch 450 is positioned in different lighting (e.g., in direct sunlight, in a shadow, partially shaded, and the like). As such, the color references 456 can act as a color index or reference point for correcting for changes in color caused by ambient light.

In certain instances, some of the color references 456 are black, others white, others red, others green, others blue. Although most of the color references 456 in FIG. 8 are shown around a perimeter of the patch (e.g., with the chemical indicators 454A-C positioned within the perimeter), other positions and arrangements of the color references 456 can be utilized in the patch 450. The overall number and the specific shape of the color references 456 (as seen from a top view) can vary from the circular or dot shape shown in FIG. 8.

Using the patches described herein, analyte concentrations can be estimated. For example, a digital image of a patch attached to a patient can be taken by a camera and an analyte concentration can be estimated based on a color of one or more chemical indicators. In certain instances, estimating the analyte concentrations involves calculating an analyte concentration for multiple chemical indicators and then applying a mathematical operation (e.g., averaging, voting) to determine the respective analyte concentrations. The analyte concentration estimations can be further based on corrections that are determined using color reference sections of the patch. Each set or grouping of chemical indicators from the digital image can be processed and their respective colors compared to a table, library, mapping, index, etc. that associates a given color of chemical indicator to a given concentration level. In certain instances, the process of estimating analyte concentrations is carried out by an application stored on and operated by a smart phone. In other instances, some or all steps can be carried out by a server or other computing system besides a smart phone that can access digital images of a patch and be programmed to determine estimated analyte concentration levels based on colors of chemical indicators shown in the digital image.

U.S. patent application Ser. No. 18/774,681 describes additional details of a wearable chemical sensing system and is herein incorporated by reference in its entirety.

### Implantable Chemical Sensor

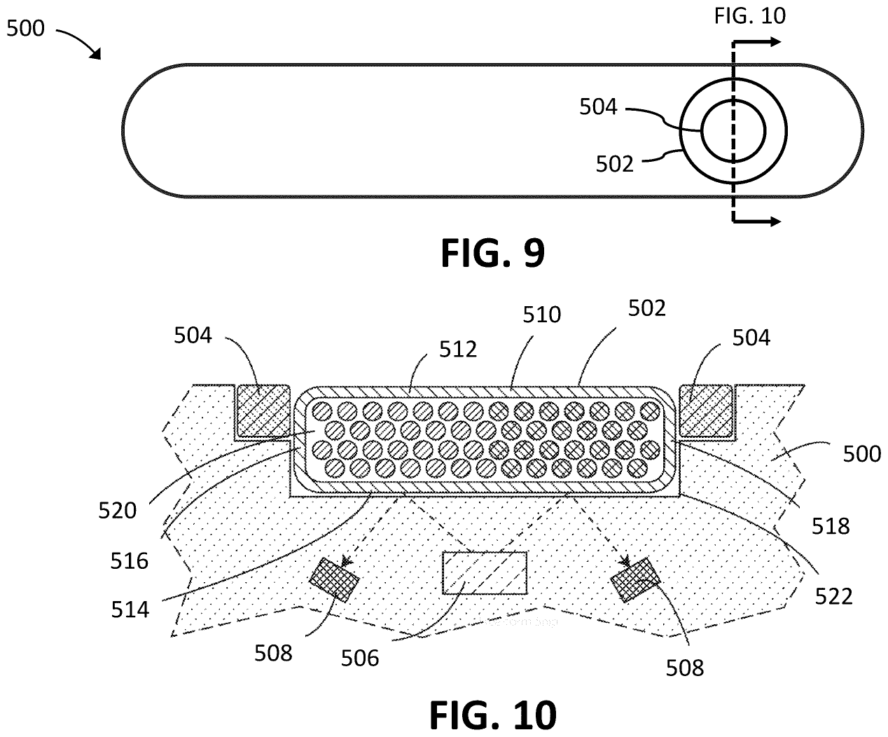

FIGS. 9 and 10 show an implantable medical device (IMD) 500 with a chemical sensor assembly.

FIG. 9 shows a top-down view of the IMD 500 with a sensing element 502 and an active agent eluting material 504 (or active agent eluting matrix) disposed around the outer perimeter of sensing element 502. In some instances, the active agent eluting material 504 forms a ring structure around the outer perimeter of sensing element 502. It will be appreciated that the sensing element 502 and the active agent eluting material 504 can have different geometric shapes and sizes.

FIG. 10 shows a cross-sectional view of a portion of the IMD 500 and its chemical sensor assembly. The IMD 500 includes an optical excitation assembly 506 and optical detection assembly 508. The sensing element 502 can include an outer barrier layer 510 formed, in full or in part, from a permeable material, such as an ion permeable polymeric matrix material. The outer barrier layer 510 can form a top 512, a bottom 514, and opposed sides 516 and 518 to surround an interior volume 520 of the sensing element 502. In certain instances, at least the top 512 of outer barrier layer 510 is permeable to sodium ions, potassium ions, hydronium ions, creatinine, urea, and the like. The outer barrier layer 510 can also include an active agent disposed therein including, but not limited to anti-inflammatory agents, angiogenic agents, and the like. The housing of the IMD 500 can include a recessed pan 522 into which the sensing element 502 fits. In some embodiments, implantable housing can define an aperture occluded by a transparent member. The transparent member can be a glass (including but not limited to borosilicate glasses), a polymer or other transparent material. The aperture can be disposed at the bottom of the recessed pan. The aperture can provide an interface allowing for optical communication between sensing element and the optical excitation and optical detection assemblies. It will be appreciated that outer barrier layer, or portions thereof, can be made from a transparent polymer matrix material to allow for optical communication between the sensing element 502 and optical excitation 506 and optical detection 508 assemblies.

The optical excitation assembly 506 can be designed to illuminate the sensing element 502. The optical excitation assembly 508 can include a light source such as a light emitting diode (LED), vertical-cavity surface-emitting lasers (VCSELs), electroluminescent (EL) devices, and the like. The optical detection assembly 508 can include a component selected from the group consisting of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like.

Various indicator beads can be positioned in the interior volume 520. The indicator beads can be used for detecting an ion concentration of a bodily fluid. For example, the indicator beads can include a polymeric support material and one or more ion selective sensing components as described more fully below. Analytes such as creatinine, potassium ion, sodium ion, hydronium ion, and the like, can diffuse through the top of the outer barrier layer and onto and/or into the indicator beads where they can bind with the ion selective sensors to produce a change in optical properties (e.g., a fluorimetric response, a colorimetric response).

U.S. Patent App. Pub. No. 2018/0344218 describes additional details of an implantable medical device with a chemical sensor assembly and is herein incorporated by reference in its entirety.

### Computing Device

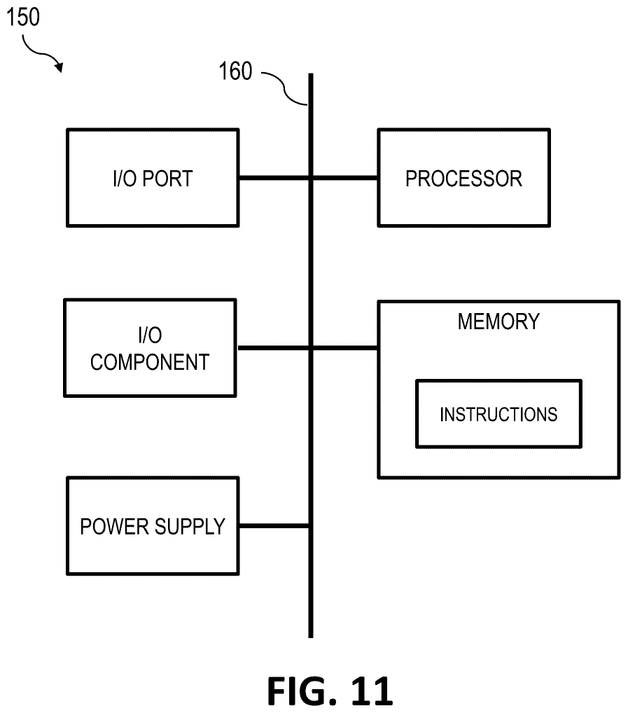

FIG. 11 is a block diagram depicting additional details of the computing device 150 shown in FIG. 3. The computing device 150 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like.

In instances, the computing device 150 includes a bus 160 that, directly and/or indirectly, couples one or more of the following devices: a processor, a memory, an input/output (I/O) port, an I/O component, and a power supply. Any number of additional components, different components, and/or combinations of components may also be included in the computing device 150.

The bus 160 represents what may be one or more busses (such as, for example, an address bus, data bus, or combination thereof). Similarly, in instances, the computing device 150 may include a number of processors, a number of memory components, a number of I/O ports, a number of I/O components, and/or a number of power supplies. Additionally, any number of these components, or combinations thereof, may be distributed and/or duplicated across a number of computing devices.

In instances, the memory includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory stores computer-executable instructions for causing the processor to implement aspects of instances of components discussed herein and/or to perform aspects of instances of methods and procedures discussed herein. The memory can comprise a non-transitory computer readable medium storing the computer-executable instructions.

The computer-executable instructions may include, for example, computer code, machine-useable instructions, and the like such as, for example, program components capable of being executed by one or more processors (e.g., microprocessors) associated with the computing device 150. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions may be configured to be executed by the processor and, upon execution, to cause the processor to perform certain processes. In certain instances, the processor, memory, and instructions are part of a controller such as an application specific integrated circuit (ASIC), field-programmable gate array (FPGA), and/or the like. Such devices can be used to carry out the functions and steps described herein.

The I/O component may include a presentation component configured to present information to a user such as, for example, a display device, a speaker, a printing device, and/or the like, and/or an input component such as, for example, a microphone, a joystick, a satellite dish, a scanner, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device including a processor, memory, and a user interface, wherein the mobile computing device is programmed to:
estimate an analyte level based on an optical property of a chemical sensor that is part of an implantable device or a wearable device,
determine an analyte response to the one or more medication dosages, and
adjust a medication dose regimen based, at least in part, on the analyte response.

2. The system of claim 1, further comprising: the wearable device with a set of needles sized for access to interstitial fluid and a chemical indicator positioned within the set of needles.

3. The system of claim 2, wherein the mobile computing device includes an image sensor.

4. The system of claim 3, wherein the mobile computing device is programmed to: determine the analyte level based, at least in part, on the optical property of the chemical indicator in a digital image.

5. The system of claim 2, wherein the chemical indicator changes optical properties in response to different potassium levels.

6. The system of claim 1, further comprising: the implantable medical device comprising a chemical indicator that changes the optical property in response to different analyte levels.

7. The system of claim 1, wherein adjusting the medication dose regimen comprises displaying a new medication dose regimen on the user interface.

8. The system of claim 1, wherein adjusting the medication dose regimen comprises changing a dosage amount of each dose.

9. A system comprising:
a mobile computing device including a processor, memory, and a user interface, wherein the mobile computing device is programmed to:
monitor analyte levels based on an optical property of a chemical sensor that is part of an implantable device or a wearable device,
determine an analyte time trend based, at least in part, on the analyte levels,
adjust a time of day for taking a medication dose based, at least in part, on the analyte time trend, and
display the time of day for taking the medication dose on the user interface.

10. A method comprising:
monitoring one or more medication dosages;
estimating an analyte level based on an optical property of a chemical sensor that is part of an implantable device or a wearable device;
determining an analyte response to the one or more medication dosages; and
adjusting a medication dose regimen based, at least in part, on the analyte response.

11. The method of claim 10, wherein the one or more medication dosages includes a first set of dosages from a first regimen and a second set of dosages from a second regimen, wherein the determining the analyte response comprises determining a difference between the analyte response to the first regimen and the second regimen.

12. The method of claim 10, wherein the one or more medication dosages involve a first type of medication, wherein the adjusting the medication dose regimen comprises adding a second type of medication.

13. The method of claim 12, wherein the second type of medication is a potassium binder medication or a potassium supplement medication.

14. The method of claim 13, wherein the analyte level is a potassium level.

15. The method of claim 10, wherein the adjusting a medication dose regimen comprises adjusting a timing between medication doses.

16. The method of claim 10, wherein the adjusting a medication dose regimen comprises adjusting a dosage amount of each dose.

17. The method of claim 10, wherein the adjusting the medication dose comprises recommending a different time of day to take the medication.

18. The method of claim 10, wherein the chemical sensor is part of the wearable device, which includes needles sized for access to interstitial fluid and a chemical indicator positioned within the needles.

19. The method of claim 18, wherein the optical property of the chemical indicator changes in response to different analyte levels.

20. The method of claim 10, wherein the chemical sensor is part of the implantable device.
